(19) United States
(12) Patent Application Publication
(10) Pub. No.: US 2012/0090624 A1
(43) Pub. Date: Apr. 19, 2012

(54) NECK SUPPORT

(76) Inventor: CHI-FU CHANG, Taichung (TW)

(21) Appl. No.: 13/241,117

(22) Filed: Sep. 22, 2011

(30) Foreign Application Priority Data
Oct. 15, 2010 (TW) 99135313
Feb. 15, 2011 (TW) 100202780

(51) Int. Cl. A61F 5/56

(52) U.S. Cl. 128/848

(57) ABSTRACT

The present invention provides a neck support including a neck wear, two support devices, and a ventilation device. The neck support is worn on human's neck. The support devices are provided on the neck wear, each of which has a jaw pad, a first tongue pad and a second tongue pad. The jaw pads are on a back of human's jaw to push human's jawbone forward, the first tongue pads are on a bottom of human's jaw to push a root of human's tongue, and the second tongue pads are on the bottom of human's jaw to push a middle of human's tongue. The jaw pads and the first tongue pads may hold human's tongue to keep the respiratory tract unobstructed, and the second tongue pads and the ventilation device help people breathing to avoid snore.

### BACKGROUND OF THE INVENTION

1. Field of the Invention

The present invention relates generally to a device to help sleeping, and more particularly to a neck support to increase sleeping quality and reduce snore.

2. Description of the Related Art

Human take about one third of lifetime in sleeping, so that sleeping is the most important thing for life. A good sleep may help health and spirit, and, on the contrary, bad sleep may cause cardiovascular diseases, apoplexy, melancholia, or other diseases.

In the past, people think snore in sleep is nothing to health but the noise. However, some researches taught us snore is an index of sleep apnea syndrome, and the sleep apnea syndrome may cause cardiovascular diseases and apoplexy.

Why people snore in sleeping? Snoring is a physiology problem. Men who snore usually have a narrow respiratory tract. In daytime, muscles of pharynx and larynx are contracted that may keep the respiratory tract open. However, when he/she lies on the bed to sleep at night, the muscles are relaxed that the tongue 90 will move toward the throat because of the gravity to narrow the respiratory tract 92, as shown in FIG. 1. Therefore, ventilation will resonate soft palate. That is snore.

There are three ways to treat snore which are: 1) Continuous-positive-airway pressure (CPAP); 2) Uvulopalatopharyngoplasty (UPPP); and 3) Mandibular advancing device (MAD). It is known that CPAP is the most effective on sleep apnea syndrome. However, there probably is no one can stand that mark, which patient has to wear it to sleep. UPPP is to cut off soft palate and uvula by surgical operation. The research found that only 50% patients no longer snore after surgery. MAD may help over 70% patients. However, people have to hold the device in the mouth that is is very uncomfortable. Sometime, people will feel pain on teeth, lower jaw, and mouth muscles because of the device.

### SUMMARY OF THE INVENTION

The primary objective of the present invention is to provide a neck support, which may reduce the snore in sleep to increase the quality of sleep.

According to the objective of the present invention, the present invention provides a neck support including a neck wear and two support devices. The neck support is worn on human's neck. The support devices are provided on the neck wear, each of which has a jaw pad and a first tongue pad. The jaw pads are on a back of human's jaw to push human's jawbone forward and the first tongue pads are on a bottom of human's jaw to push a root of human's tongue.

In an embodiment, the support device further includes a second tongue pad on the bottom of human's jaw to push a middle of human's tongue.

In an embodiment, the neck wear is provided with a neck pad and two shoulder pads.

In an embodiment, the present invention further provides a ventilation device.

The jaw pads and the first tongue pads may hold human's tongue to keep the respiratory tract unobstructed, and the second tongue pads and the ventilation device help people breathing to avoid snore.

### BRIEF DESCRIPTION OF THE DRAWINGS

FIG. 1 is a sketch diagram of why people snore;

FIG. 2 is a perspective view of a preferred embodiment of the present invention;

FIG. 3 is an exploded view of the preferred embodiment of the present invention;

FIG. 4 is a sectional view of the preferred embodiment of the present invention, showing fastener releasing the support device;

FIG. 5 is a sectional view of the preferred embodiment of the present invention, showing fastener securing the support device;

FIG. 6 is a sketch diagram of the preferred embodiment of the present invention, showing the positions of the jaw pad, first tongue pad, and the second tongue pad;

FIG. 7 is a perspective view of the ventilation device of the preferred embodiment of the present invention;

FIG. 8 is a perspective view of the second ventilation device of the preferred embodiment of the present invention; and

FIG. 9 is a perspective view of the third ventilation device of the preferred embodiment of the present invention.

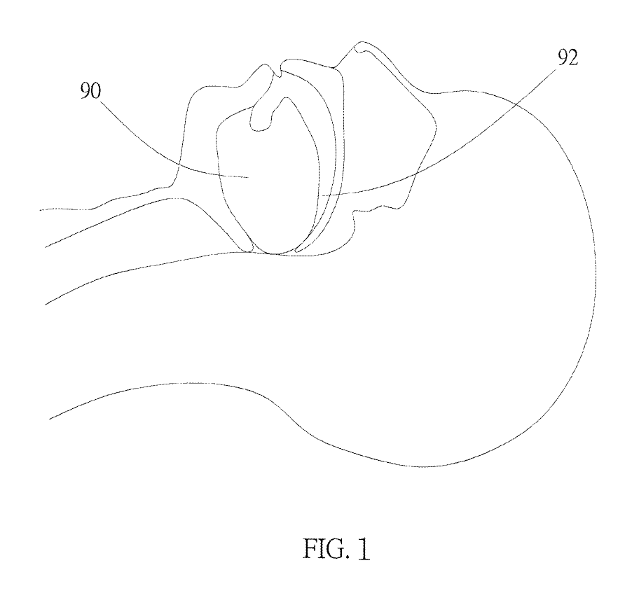

### DETAILED DESCRIPTION OF THE INVENTION

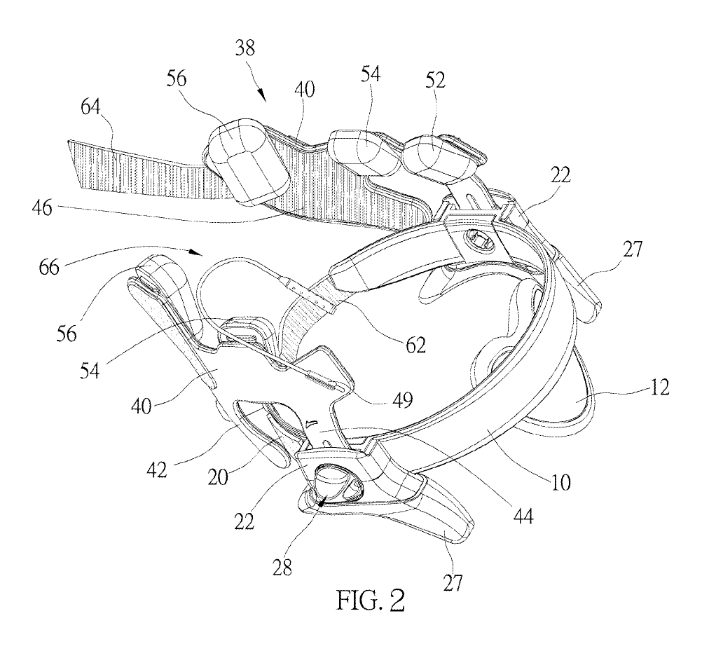

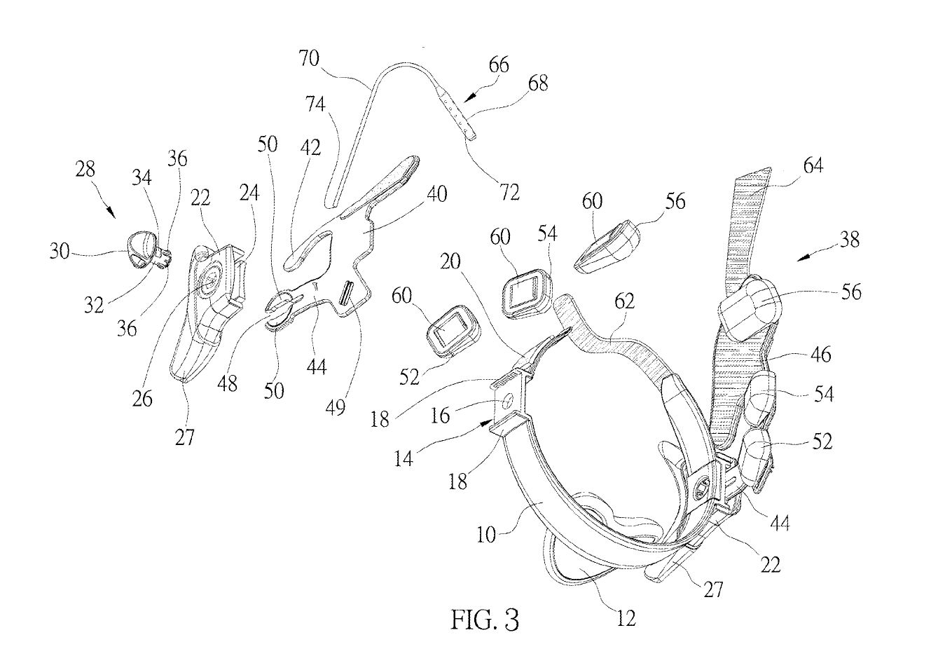

As shown in FIG. 2 and FIG. 3, a neck support of the preferred embodiment of the present invention includes a neck wear 10, two connector 22, two support devices 38, and a ventilation device 66.

The neck wear 10 is a C-ring made of plastic or other flexible materials. The neck wear 10 has a neck pad 12 at middle of an inner side, and two connecting portions 14 at opposite sides of the neck pad 12. The neck pad 12 has a curved surface made of silicon rubber or other flexible materials to fit the curvature of the back of human's neck. Each connecting portion 14 has a bore 16 and two sidewalls 18 on opposite sides of the bore 16. Teeth are provided on the sidewalls 18 and face each other. The neck wear 10 is provided with a hook member 20 of Velcro beside the connecting portions 14 respectively.

Each connector 22 has a longitude slot 24 and a transverse through hole 26. The connectors 22 engage the connecting portions 14 of the neck wear 10 that the sidewalls 18 are in the slot 24, and the bore 16 are aligned with the through hole 26. Each connector 22 has a shoulder pad 27 at a bottom thereof. Two fasteners 28 are provided in the through holes 26 of the connectors 22. The fastener 28 is a bolt-like member, having a head portion 30 and a shaft 32. The shaft 32 has two indentations 34 at opposite sides that a short diameter L1 is formed between the indentations 34, and a long diameter L2 is perpendicular to the short diameter L1. The long diameter L2 is greater than the short diameter L1. The shaft 32 has gaps at a tail to form several flexible pieces 36. The shaft 32 is inserted into the through hole 26 of the connector 22 and the bore 16 of the connecting portion 14 of the neck wear 10 with the tail end going out of the bore 16 that the flexible pieces 36 may engage the connecting portion 14 to hold the shaft 32 in the bore 16 and the through hole 26. The head portion 30 is left out of the bore 16 of the connector 22 that user may hold the head portion 30 to turn the fastener 28.

Each support device 38 has a base 40. The base 40 has an extending portion 42 and a connecting portion 44. At an inner side of the base 40 (including the extending portion 42) is provided with a loop member 46 of Velcro. The connecting portion 44 has a gap 48 and two flexible arms 50 at opposite sides of the gap 48. A width of the gap 48 is greater than short diameter L1 of the shaft 32 of the fastener 28 and is shorter than long diameter L2. Each flexible arm 50 is provided with teeth on an outer convex edge. The support device 38 further has a holder 49 on an outer side.

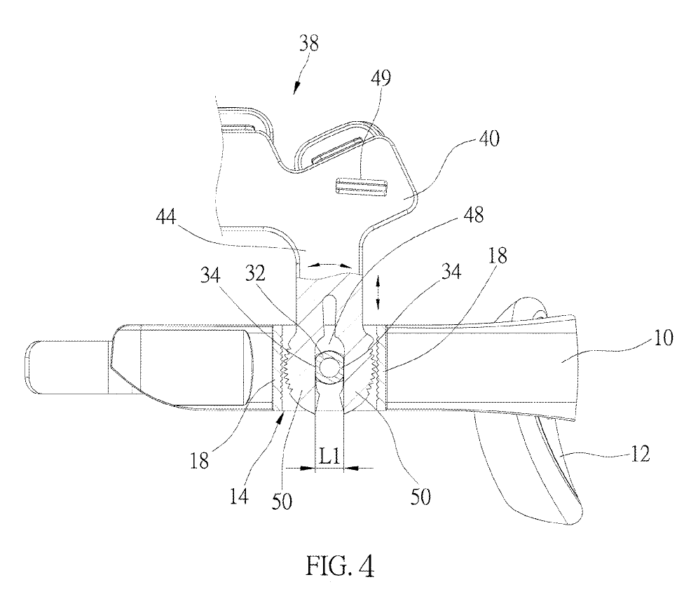

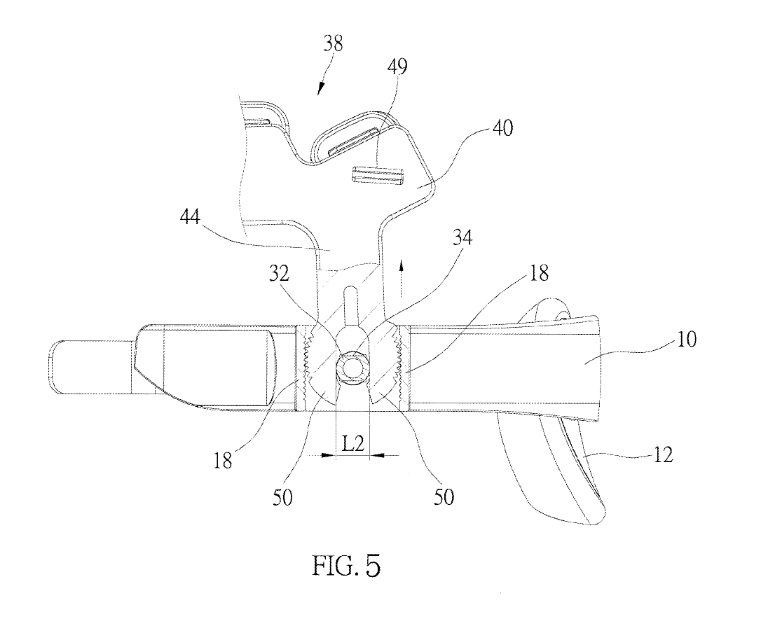

As shown in FIG. 4 and FIG. 5, the connecting portions 44 of the support devices 38 are inserted into the slots 24 of the connectors 22 respectively that the shafts 32 of the fasteners 28 are in the gaps 48, and the teeth on the flexible arms 50 are in association with the teeth on the sidewalls 18. As shown in FIG. 4, when one turn the fastener 28 to have the indentations 34 facing the flexible arms 50, the teeth of the flexible arms 50 and the sidewalls 18 are disengaged that the support device 38 is free to move along the gap 48 and rotate along the shaft 32 of the fastener 28. As shown in FIG. 5, when one turns the fastener 28 to have the indentations 34 off the flexible arms 50, the shaft 32 will move the flexible arms 50 outwardly to engage the teeth on he flexible arms 50 and the sidewalls 18 that the support device 38 is fastened and cannot be moved or turned. At the same time, one may connect the loop member 46 on the extending portion 42 to the hook member 20 on the neck wear 10 to form a second connection, except for the connecting portion 44, of the support device 38 and the neck wear 10 that may firmly fasten the supporting devices 38.

Each support device 38 is provided with a jaw pad 52, a first tongue pad 52, and a second tongue pad 54 on the inner side of the base 40. The jaw pad 52, the first tongue pad 52, and the second tongue pad 54 have a flexible surface at an outer side to touch human's body and are provided with a hook member of Velcro 60 at an inner side to be mounted on predetermined positions of the inner side of the base 40, on which the loop member of Velcro 46 is provided.

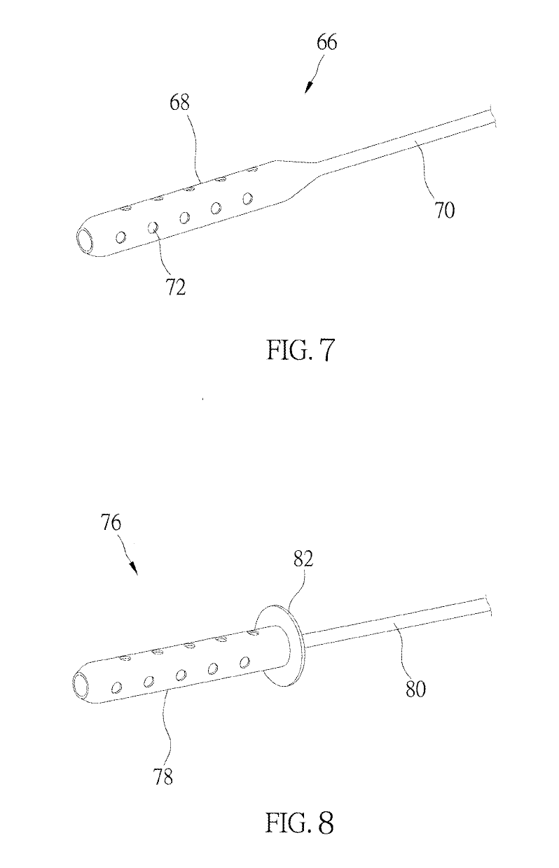

As shown in FIG. 7, the ventilation device 66 is a flexible tubular member with a main section 68 and an extending section 70. A diameter of the main section 68 is greater than that of the extending section 70, and the extending section 70 is longer than the main section 68. The ventilation device 66 has several bores 72 on the main section 68 and an opening 74 on a tail end of the extending section 70. The extending section 70 is bent, and is engaged to the holder 49 on the support device 38 that user may hold the main section 68 in the mouth.

One may wear the neck wear 10 on the neck with the neck pad 12 against the back of the neck and the shoulder pads 27 on the shoulders, and then fastens it by a belt 62 (with Velcro). Next, one may adjust the support devices 38 to locate the jaw pads 52, the first tongue pads 54, and the second tongue pads 56 at predetermined positions. Finally, put the ventilation device 66 in the mouth.

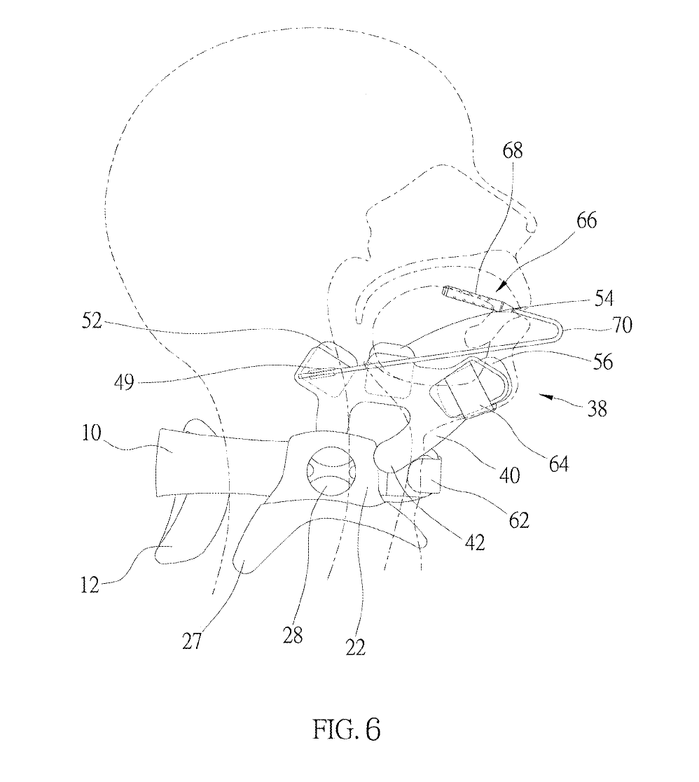

As shown in FIG. 6, the jaw pads 52 press two back ends of human's jawbone to push the lower jaw forward, as well as the tongue, to hold the tongue when one lies on bed. The first tongue pads 54 are on a middle section of a bottom of the lower jaw to hold the root of the tongue directly. The jaw pads 52 and the first tongue pads 54 may hold the tongue to prevent it from dropping into the throat when one lies on bed, and, therefore, the snore will be stopped. The second tongue pads 56 are on a front section of the bottom of the lower jaw to push left and right sides of the tongue that the tongue is kept in a U shape. The second tongue pads 56 and the ventilation device 66 may help breathing in sleep.

In addition, people usually open the mouth in sleep. This is because the muscles around the mouth relax when one lies on bed and sleep. As a result, one breaths by mouth in sleep, and it may cause the throat dry, pain, and inflammation. When one wear the neck support of the present invention to sleep, the support devices 38 may be bent into a circle and fastened by a belt 64 (with Velcro). It may enhance the functions of the jaw pads 52, the first tongue pads 54, and the second tongue pads 56 to keep the mouth close in sleep. It may avoid the problem of mouth open as described above.

People usually turn the head in sleep. When he/she wears the neck support of the present invention to sleep and turn the head, it will exert the shoulder pad 27 on shoulder through the jaw pad 52 to massage shoulder.

The main feature of the present invention is the pads, such as the jaw pads 52, the first tongue pads 54, and the second tongue pads 56, to support specific muscles and bones that the tongue will be hold to avoid snoring.

The present invention further provides the neck pad and the shoulder pads to keep the neck straight in sleep. It will reduce the musculoskeletal injury of neck in sleep.

In hospital, some patients with respiratory tract jam problem have to wear oxygen mask. The oxygen mask provides air with a predetermined pressure, and that may hurt lung. If such patients wear the neck support of the present invention, it may get the same therapy with lower pressure of air that may reduce the risk of hurting lung.

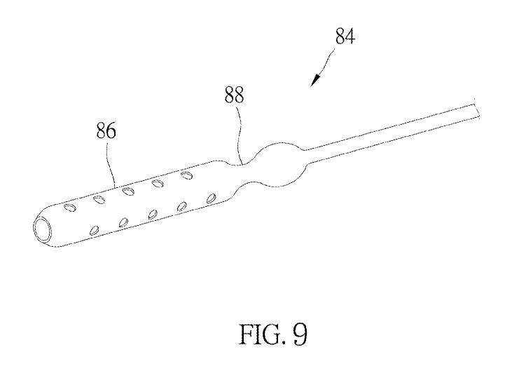

FIG. 8 shows another ventilation device 76, which has a main section 78, an extending section 80, and a rim 82 between the main section 78 and the extending section 80. The rim 82 will be stopped by lips to prevent the main section 80 from dropping into the throat. FIG. 9 show a third ventilation device 84, which provides a annular recess 88 on a main 86 to be hold by lips.

The description above is a few preferred embodiments of the present invention and the equivalence of the present invention is still in the scope of claim construction of the present invention.

What is claimed is:

1. A neck support, comprising:
a neck wear to be worn on human's neck; and
two support devices provided on the neck wear, each of which has a jaw pad on a back of human's jaw to push human's jawbone forward and a first tongue pad on a bottom of human's jaw to push a root of human's tongue.

2. The neck support as defined in claim 1, wherein the support device further having a second tongue pad on the back of human's jaw to push a middle of human's tongue.

3. The neck support as defined in claim 1, further comprising two fasteners engaging the support devices, wherein each of the support devices has a flexible arm with teeth, and the neck wear is provided with teeth in association with the teeth of the flexible arm, the fastener moves the flexible arm to engage the teeth of the flexible arm and the neck wear.

4. The neck support as defined in claim 1, further comprising two fasteners engaging the support devices, wherein each of the support devices has a gap and two flexible arms, each of which has teeth, on opposite sides of the gap, and the neck wear is provided with teeth in association with the teeth of the flexible arms, the fastener passes through the gap of the support device, further wherein the fastener has a short diameter smaller than a width of the gap and a long diameter greater than the width of the gap that the fastener is turn to move the flexible arms to engage and disengage the teeth of the flexible arms and the neck wear.

5. The neck support as defined in claim 3, wherein the flexible arm has a convex edge, on which the teeth are provided.

6. The neck support as defined in claim 4, wherein each of the flexible arms has a convex edge, on which the teeth are provided.

7. The neck support as defined in claim 4 wherein the fastener has at least an indentation to form the short diameter.

8. The neck support as defined in claim 1, further comprising a neck pad on the neck wear to support a back of human's neck.

9. The neck support as defined in claim 1, further comprising two shoulder pads on the neck wear to rest on human's shoulder.

10. The neck support as defined in claim 1, further comprising a belt to connect the support devices.

11. The neck support as defined in claim 1, wherein the support device has a connector to engage the neck wear and an extending portion to detachably connect the neck wear.

12. The neck support as defined in claim 1, further comprising a ventilation device including a main section with at least a bore to be hold in human's mouth and an extending section with an opening, which is communicated with the bore, to be mounted on the support device.

13. The neck support as defined in claim 12, wherein the ventilation device further has a rim between the main section and the extending section.

14. The neck support as defined in claim 12, wherein the ventilation device further has a recess on the main section.
